(19) United States
(12) Patent Application Publication
(10) Pub. No.: US 2009/0234186 A1
(43) Pub. Date: Sep. 17, 2009

(54) Gastrointestinal endoscope with deflectable direction-change structure and deflectable direction-change tube thereof

(76) Inventors: Chun-Ming Lin, Hsinchu (TW); Shih-Chieh Lu, Hsinchu (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/379,257

(22) Filed: Feb. 18, 2009

(30) Foreign Application Priority Data
Mar. 14, 2008 (TW) 97109170

(51) Int. Cl. A61B 1/273

(52) U.S. Cl. 600/113

(57) ABSTRACT

Disclosed is a gastrointestinal endoscope, including a deflectable direction-change tube, which consists of plural ring units, each having a hollow section. Adjacent ring units are spaced by a predetermined distance, having opposing end faces between which at least two integrally formed connectors are provided to connect the adjacent ring units together. An end of the deflectable direction-change tube is operatively coupled to a direction-change operation device and an opposite end coupled to a capsule endoscope clamping structure. At least one control wire extends through a bore of the deflectable direction-change tube and connected between the direction-change operation device and the capsule endoscope clamping structure. By operating an operation capstan to have the control wire pulling the capsule endoscope clamping structure, the deflectable direction-change tube is controlled to change direction, thereby changing direction of a capsule endoscope retained by the capsule endoscope clamping structure.

### FIELD OF THE INVENTION

The present invention relates to a gastrointestinal endoscope, and in particular to a gastrointestinal endoscope having a deflectable direction-change structure and a deflectable direction-change tube used in the gastrointestinal endoscope.

### BACKGROUND OF THE INVENTION

A gastrointestinal endoscope has been long used in examine gastrointestinal diseases. With constant improvement, the gastrointestinal endoscope is now a necessary diagnosis tool for medical doctors. Beside the function of diagnosis, the application of the gastrointestinal endoscope in therapy has been expanded widely, of which one of the purposes of the development and application of the gastrointestinal endoscope is to reduce the pain of the patients, to improve the diagnosis performance, and to enhance the process of treatment.

The gastrointestinal endoscope is divided as upper gastrointestinal endoscopy, enterscopy, endoscopic retrograde cholangiopancreatography, and lower gastrointestinal endoscopy. In doing regular examination by using the gastrointestinal endoscope, a capsule endoscope mounted at a front tip of the gastrointestinal endoscope must be properly controlled for direction change.

### SUMMARY OF THE INVENTION

A conventional gastrointestinal endoscope uses a helical spring of a predetermined length to serve as a deflectable direction-change tube, which, when cooperating with a direction-change operation device and a control pull wire, may effect the control of direction change for the gastrointestinal endoscope. Such a conventional device, although effective in controlling direction change, is disadvantageous in that the manufacturing of the helical spring based deflectable direction-change tube and mounting related parts to front and rear ends thereof are both difficult. A capsule endoscope is recently developed and overcomes the long existing problem of lacking a proper tool for performing examination of small intestines. Thus, it is desired to combine the operability of the conventional endoscope and the function of examination of small intestines provided by the capsule endoscope to provide a total and effective diagnosis tool for digestive tract.

Thus, an objective of the present invention is to provide a deflectable direction-change tube structure applicable to a gastrointestinal endoscope, wherein the deflectable direction-change tube is comprised of a plurality of ring units that each has a hollow section and integrally formed connectors.

Another objective of the present invention is to provide a gastrointestinal endoscope that has a deflectable direction-change structure, wherein a deflectable direction-change tube in accordance with the present invention is operatively coupled to a direction-change operation device, a capsule endoscope clamping structure, and a control wire to enhance the operation controllability of the gastrointestinal endoscope.

The solution adopted in the present invention to overcome the problems of the conventional techniques is to provide a tubular body that comprises a plurality of ring units that each has a hollow section and connectors that are provided between opposing end faces of adjacent ring units and integrally formed with the ring units to connect the ring units together. When a deflection force is applied to the deflectable direction-change tube, the ring units are direction changeable by rotation about the connectors and a maximum direction-changeable angle of the deflectable direction-change tube is defined as the end faces of the adjacent ring units are brought into contact with each other. Opposite ends of the deflectable direction-change tube are respectively coupled to a direction-change operation device and a capsule endoscope clamping structure and control wires. By operating the direction-change operation device to have the control wire pulling the capsule endoscope clamping structure, the deflectable direction-change tube is controlled by a pulling force applied by the control wire to change direction, thereby changing direction of a capsule endoscope retained by the capsule endoscope clamping structure.

The solution of the present invention allows combination with a capsule endoscope and full use of the function of deflection for direction change to control the direction. Thus, the gastrointestinal endoscope of the present invention offers the same excellent direction change controllability as that provided by the deflectable direction-change tube. The manufacturing of the deflectable direction-change tube can be made through integral forming techniques of flexible materials, such as rubber materials so as to have the advantages of being easy to manufacture, being easy to control the structural properties, and excellent direction change controllability.

Further, the deflectable direction-change tube constructed in accordance with the present invention has the function of change direction in given orientation, such as between two directions or among four directions. And, since the distance between adjacent ring units imposes a limitation to the maximum direction-change angle, in practical applications, excellent operability and enhanced controllability can be realized.

### BRIEF DESCRIPTION OF THE DRAWINGS

The present invention will be apparent to those skilled in the art by reading the following description of the preferred embodiments of the present invention, with reference to the attached drawings, in which:

FIG. 1 is a perspective view of a first embodiment of the present invention;

FIG. 2 is another perspective view of the first embodiment of the present invention;

FIG. 3 is a side elevational view of a deflectable direction-change tube in accordance with the present invention;

FIG. 4 is a cross-sectional view taken along line 4-4 of FIG. 2;

FIG. 5 is a view schematically illustrating deflection of the deflectable direction-change tube when a deflection force is applied to the deflectable direction-change tube;

FIG. 6 is another view schematically illustrating deflection of the deflectable direction-change tube when a deflection force is applied to the deflectable direction-change tube;

FIG. 7 is a further view schematically illustrating deflection of the deflectable direction-change tube when a deflection force is applied to the deflectable direction-change tube;

FIG. 8 is a perspective view of a deflectable direction-change tube in accordance with a second embodiment of the present invention;

FIG. 9 is a side elevational view of the deflectable direction-change tube of the second embodiment of the present invention; and

FIG. 10 is another side elevational view of the deflectable direction-change tube of the second embodiment of the present invention.

### DETAILED DESCRIPTION OF THE PREFERRED EMBODIMENTS

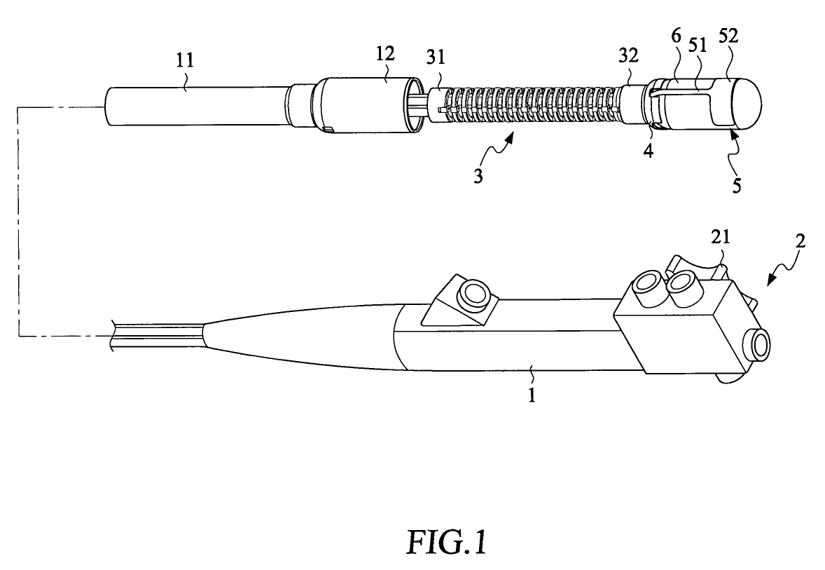

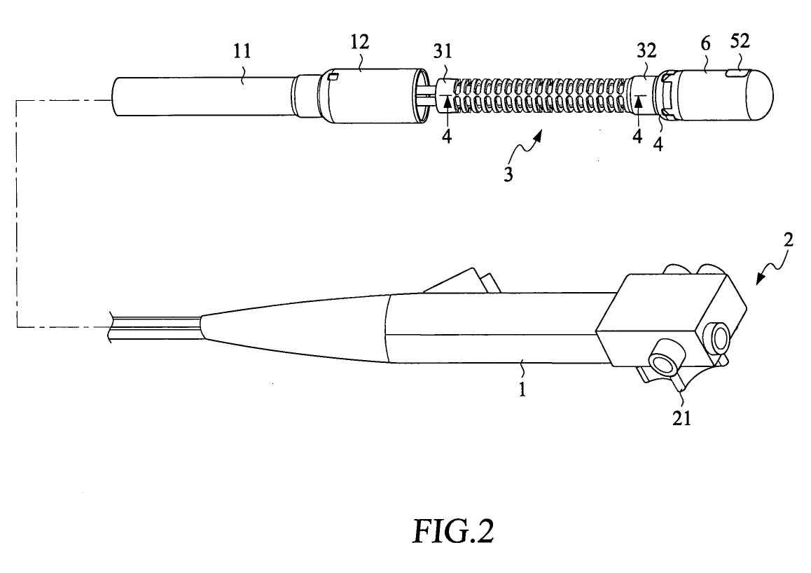

With reference to the drawings and in particular to FIGS. 1 and 2, of which FIG. 1 is a perspective view of the present invention and FIG. 2 is another perspective view of the present invention, the present invention provides a gastrointestinal endoscope that has a deflectable direction-change structure. The gastrointestinal endoscope of the present invention comprises a handle 1 and a direction-change operation device 2 coupled to a rear end of the handle 1. The direction-change operation device 2 comprises an operation capstan 21.

A deflectable direction-change tube 3, which is made in the form of a tubular structure, has a first end 31 and a second end 32. The first end 31 is attached to a front end of the handle 1 and is operatively coupled to the direction-change operation device 2, and the second end 32 is coupled to a capsule endoscope clamping structure 4.

The capsule endoscope clamping structure 4 has a front end that shows a clamp-like structure. A flexible circuit board 5 extends from the front end of the capsule endoscope clamping structure 4. The flexible circuit board 5 comprises an extension section 51 that extends from the capsule endoscope clamping structure 4 and an attaching section 52.

The front end of the capsule endoscope clamping structure 4 functions to clamp a capsule endoscope 6, which has a circumferential surface on which a plurality of electrical contacts is formed. The attaching section 52 of the flexible circuit board 5 is attached to the circumferential surface of the capsule endoscope 6 in such a way that electrical contacts (not shown) formed on the attaching section 52 are respectively set in contact engagement with the corresponding contacts formed on the circumferential surface of the capsule endoscope 6.

To assemble the components/parts of the gastrointestinal endoscope of the present invention, a direction-change mechanism sleeve 11 is fit over and encloses the deflectable direction-change tube 3 and an outer, capsule-housing plastic cover 12 is set to enclose the capsule endoscope 6.

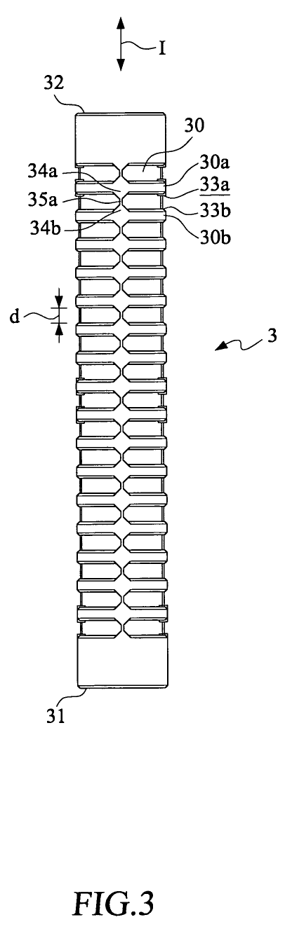

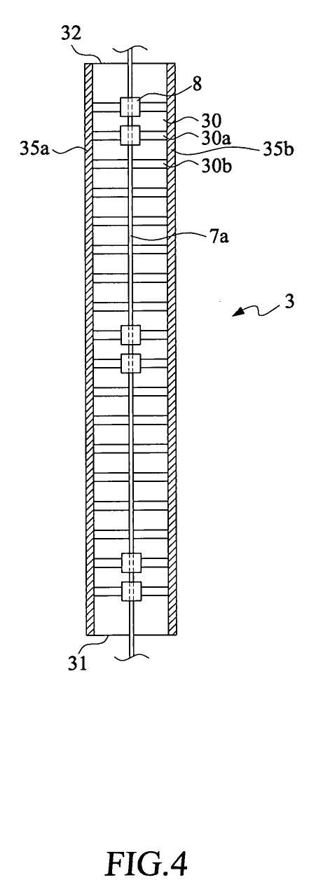

Also referring to FIG. 3, which shows a side elevational view of the deflectable direction-change tube 3, and FIG. 4, which shows a cross-sectional view taken along line 4-4 of FIG. 2, the deflectable direction-change tube 3 is made of hollow tubular body that is deflectable for change of direction and is comprised of a plurality of ring units having a hollow portion, such as those indicted by reference numerals 30a, 30b, which are arranged along an axial direction I of the tubular body to make a tubular body having a first end 31 and a second end 32. The hollow portions of the ring units 30a, 30b together define a through-extending bore 30. A predetermined distance d is set between adjacent ring units 30a, 30b. The ring units 30a, 30b have ring end faces 33a, 33b corresponding to each other and each forming at least two jointing sections 34a, 34b. A connector 35a, 35b, which is preferably integrated with the respective ring unit 30a, 30b, connects between corresponding jointing sections so as to connect the adjacent ring units 30a, 30b together to form the hollow tubular body.

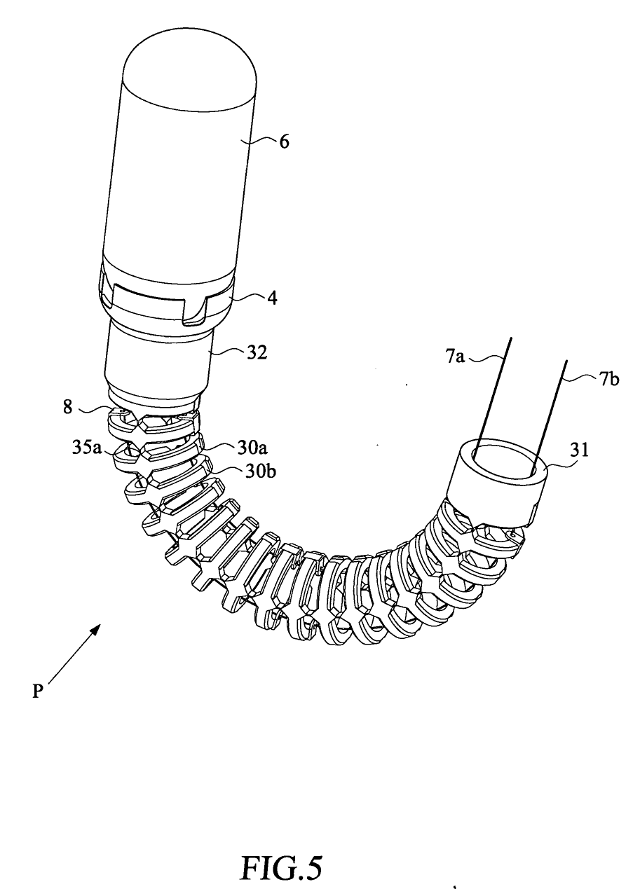

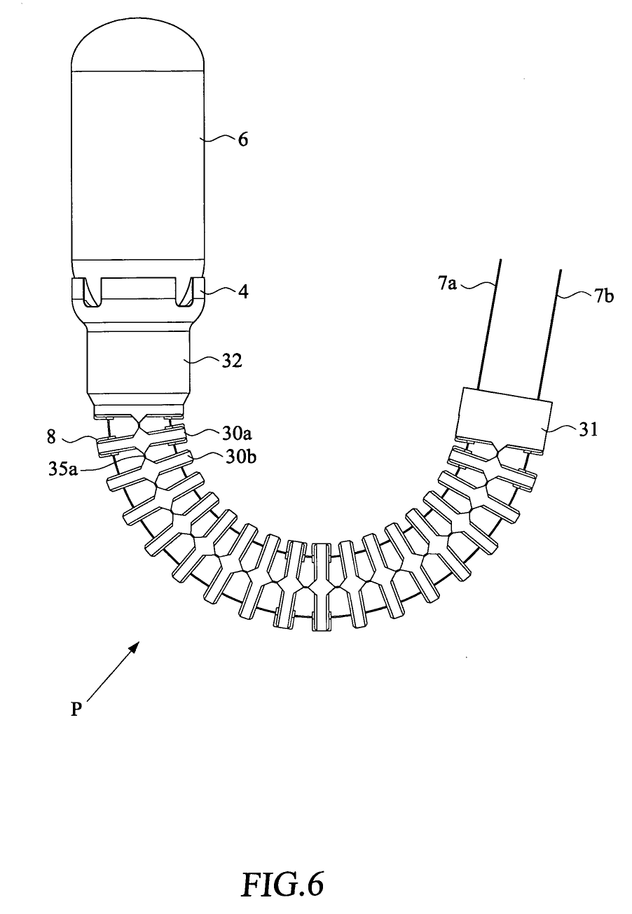

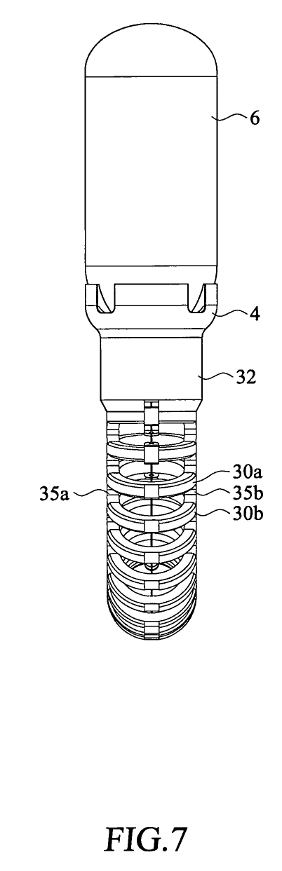

Referring to FIGS. 5-7, when a deflection force P is applied to the deflectable direction-change tube 3, the direction of each ring unit 30a, 30b is changed by being rotated about the connectors 35a, 35b that serve as rotation centers. A maximum deflection angle of the deflectable direction-change tube 3 is defined as the ring end faces 33a, 33b of the adjacent ring units 30a, 30b are brought into contact with each other.

In practice, two control wires 7a, 7b are set inside the deflectable direction-change tube 3. The two control wires 7a, 7b are securely fixed to an inside circumferential wall of each ring unit 30a, 30b by control wire clips 8 (see FIG. 4) and the control wires 7a, 7b are set to extend through the bore 30 of the deflectable direction-change tube 3 to respectively couple between the operation capstan 21 of the direction-change operation device 2 and the capsule endoscope clamping structure 4.

To operate, the operation capstan 21 of the direction-change operation device 2 is turned and one of the control wires 7a, 7b is driven to pull the capsule endoscope clamping structure 4 to induce the deflection force P applied to the deflectable direction-change tube 3, whereby the deflectable direction-change tube 3 is controlled by the pulling force applied by the control wire 7a, 7b to change direction and thus, changing the direction of the capsule endoscope 6.

The deflectable direction-change tube 3 can be made of flexible material, such as rubber materials, by using integral forming techniques to form the ring units 30a, 30b and the connectors 35a, 35b connecting between adjacent ring units 30a, 30b.

In the previously discussed embodiment, the deflectable direction-change tube 3 is made so as to present, between adjacent ring units 30a, 30b, two connectors 35 that are preferably angularly spaced approximately 180 degrees and thus substantially diametrically opposite to each other, whereby once subjected to the deflection force, the deflectable direction-change tube 3 is allowed to provide the function of direction change between two directions, which in this case, are opposite to each other.

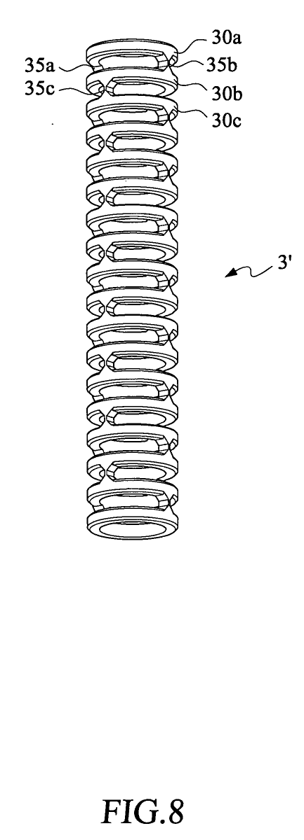

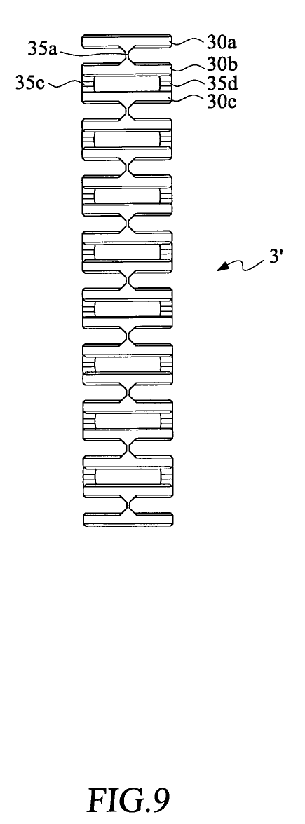

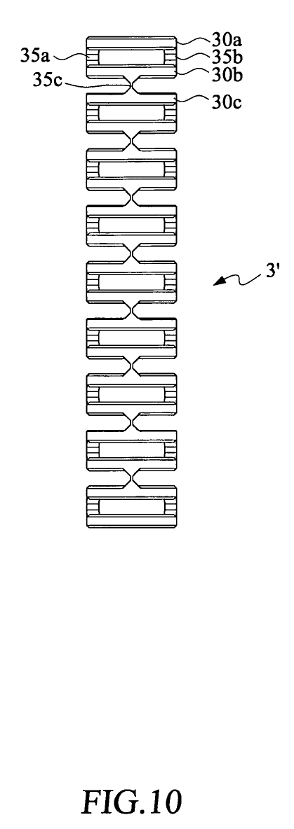

The same solution is also applicable to a deflectable direction-change tube having a function of direction change among four directions or more directions. For example, as shown in FIGS. 8-10, a second embodiment of the deflectable direction-change tube 3 in accordance with the present invention is shown. In the instant embodiment, the deflectable direction-change tube, which is designated by reference numeral 3′ for distinction, comprises two adjacent ring units 30a, 30b between which 180-degree spaced connectors 35a, 35b are provided and further, two additional connectors 35c, 35d, which are also angularly spaced by approximately 180 degrees, are arranged between the ring unit 30b and a next ring unit 30c. The additional connectors 35c, 35d and the connectors 35a, 35b are angularly offset with respect to each other by 90 degrees so that a 90 degree difference in angular position is present between each additional connector 35c, 35d and an adjacent one of the connectors 35a, 35b. In this way, the deflectable direction-change tube 3′ is made direction changeable among four directions.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A gastrointestinal endoscope, comprising:
a deflectable direction-change tube, comprising a tubular body structure having first and second ends, the deflectable direction-change tube forming a through-extending bore, the deflectable direction-change tube comprising a plurality of ring units each having a hollow section and arranged in such a way that adjacent ring units are spaced by a predetermined distance and the adjacent ring units have opposing end faces between which at least two connectors are provided to connect the adjacent ring units together so that the ring units are direction changeable by rotation about the connectors;
a direction-change operation device, operatively coupled to the first end of the deflectable direction-change tube;
a capsule endoscope clamping structure, coupled to the second end of the deflectable direction-change tube and functioning to clamp a capsule endoscope; and
at least one control wire, extending through the bore of the deflectable direction-change tube and connected between the direction-change operation device and the capsule endoscope clamping structure;
wherein by operating the direction-change operation device to drive the control wire to pull the capsule endoscope clamping structure, the deflectable direction-change tube is controlled by a pulling force applied by the control wire to change direction, thereby changing direction of the capsule endoscope.

2. The gastrointestinal endoscope as claimed in claim 1, wherein at least one of the ring units of the deflectable direction-change tube comprises a control wire clip that securely fixes the control wire that extends through the through-extending bore to an inside circumferential wall of the ring unit.

3. The gastrointestinal endoscope as claimed in claim 1, wherein the two connectors that are provided between the end faces of the adjacent ring units are set opposite to each other by being angularly spaced from each other by 180 degrees.

4. The gastrointestinal endoscope as claimed in claim 1, wherein the two connectors that are provided between the end faces of the adjacent ring units are set opposite to each other by being angularly spaced from each other by 180 degrees, and wherein two additional connectors are provided for a next adjacent ring unit and are set opposite to each other by being angularly spaced from each other, the additional connectors and said at least two connectors being arranged to angularly offset by 90 degrees.

5. The gastrointestinal endoscope as claimed in claim 1, wherein a flexible circuit board is arranged between the capsule endoscope clamping structure and the capsule endoscope to provide signal connection therebetween.

6. The gastrointestinal endoscope as claimed in claim 5, wherein the flexible circuit board comprises an extension section that extends from the capsule endoscope clamping structure and an attaching section that is attached to a circumferential surface of the capsule endoscope with contacts formed on the attaching section brought into engagement with contacts set on the circumferential surface of the capsule endoscope.

7. A deflectable direction-change tube comprising a hollow tubular body that is deflectable for direction change, comprising a plurality of ring units that has hollow portions and is distributed along an axial direction of the tubular body to form a tubular body structure having first and second ends, the hollow portions of the ring units together defining a through-extending bore, adjacent ring units being spaced by a predetermined distance, the adjacent ring units having opposing end faces each forming at least two jointing sections, which are respectively provided with connectors integrally formed with the ring units to connect the adjacent ring units together to form the hollow tubular body, whereby when a deflection force is applied to the deflectable direction-change tube, the ring units are direction changeable by rotation about the connectors, a maximum deflection angle of the deflectable direction-change tube being defined as the opposing end faces of the adjacent ring units are brought into contact with each other.

8. The deflectable direction-change tube as claimed in claim 7, wherein the first end of the deflectable direction-change tube is operatively coupled to a direction-change operation device and the second end is coupled to a capsule endoscope clamping structure, the direction-change operation device being connected to the capsule endoscope clamping structure by at least one control wire that extends through the through-extending bore defined by the ring units.

9. The deflectable direction-change tube as claimed in claim 8, wherein at least one of the ring units of the deflectable direction-change tube comprises a control wire clip that securely fixes the control wire that extends through the through-extending bore to an inside circumferential wall of the ring unit.

10. The deflectable direction-change tube as claimed in claim 8, wherein the two connectors that are provided between the end faces of the adjacent ring units are set opposite to each other by being angularly spaced from each other by 180 degrees.

11. The deflectable direction-change tube as claimed in claim 7, wherein the two connectors that are provided between the end faces of the adjacent ring units are set opposite to each other by being angularly spaced from each other by 180 degrees, and wherein two additional connectors are provided for a next adjacent ring unit and are set opposite to each other by being angularly spaced from each other, the additional connectors and said at least two connectors being arranged to angularly offset by 90 degrees.
